(19) United States
(12) Patent Application Publication
(10) Pub. No.: US 2022/0354543 A1
(43) Pub. Date: Nov. 10, 2022

(54) MEDICAL STABILIZATION APPARATUS

(71) Applicant: BE Innovative Medical Solutions, LLC, Denver, CO (US)

(72) Inventors: Michael Ryan Ballard, Denver, CO (US); Aaron Treat, Denver, CO (US)

(21) Appl. No.: 17/662,771

(22) Filed: May 10, 2022

Related U.S. Application Data
(60) Provisional application No. 63/186,426, filed on May 10, 2021.

(51) Int. Cl. A61B 17/70; A61G 13/10

(52) U.S. Cl. CPC A61B 17/7007; A61G 13/101

(57) ABSTRACT

Systems and methods may provide medical stabilization according to one or more aspects of the present disclosure. For example, a medical stabilization apparatus may stabilize a spine of a patient during a medical procedure. An apparatus may include a tube component, a cable (e.g., a cable running through the tube component), an end assembly (e.g., for adjusting tension of the cable), a clamp adapter component (e.g., to attach the cable/tube component to another medical device, such as a spinous process clamp), and a sheath component. The sheath component may be secured over tube components, and the sheath component may attach to a channel of a channel component (e.g., to secure the tube components to a base or a platform).

### CROSS-REFERENCE TO RELATED APPLICATION

This application claims priority from Provisional Patent Application No. 63/186,426 filed on May 10, 2021, in the United State Patent and Trademark Office, and all the benefits accruing therefrom under 35 U.S.C. 119, the contents of which in their entirety are herein incorporated by reference.

### BACKGROUND

The following relates generally to medical devices, and more specifically to systems and methods for stabilizing and securing a patient.

It is often desirable to stabilize or secure a patient, or parts of a patient's body during a medical procedure. In some cases, movement of a patient during a medical procedure can causes errors in the procedure or harm to the patient. Therefore, there is a need in the art for systems and methods of securing and stabilizing a patient during a medical procedure.

### SUMMARY

An apparatus, system, and method for stabilizing and securing a patient during medical procedures are described. One or more aspects of the apparatus, system, and method include a tube component; a cable extending through the tube component; a clamp adapter component attached to a first end of the tube component, wherein the clamp adapter component comprises a clamp attachment part configured to attach the clamp adapter to a spinal clamp; an end assembly attached to a second end of the tube component, wherein the end assembly comprises a tension adjustment part configured to adjust a tension of the cable; and a sheath component surrounding a portion of the tube component, wherein the sheath component comprises a position locking mechanism configured to attach the sheath component to a base component.

An apparatus, system, and method for stabilizing and securing a patient during medical procedures are described. One or more aspects of the apparatus, system, and method include a plurality of tube components; a plurality of cables, wherein each of the plurality of cables extends through a corresponding tube component of the plurality of tube components; a clamp adapter component attached to a first end of each of the plurality of tube components, wherein the clamp adapter component comprises a clamp attachment part; a plurality of end assemblies attached to a second end of each of the plurality of tube components, respectively, wherein each of the plurality of end assemblies comprises a tension adjustment part configured to adjust a tension of a corresponding cable of the plurality of cables; and a plurality of sheath components surrounding a portion of each of the plurality of tube components, respectively, wherein each of the plurality of sheath components comprises a position locking mechanism.

A method, apparatus, non-transitory computer readable medium, and system for stabilizing and securing a patient during medical procedures are described. One or more aspects of the method, apparatus, non-transitory computer readable medium, and system include providing a tube component of an apparatus; providing a cable extending through the tube component of the apparatus; providing a clamp adapter component of the apparatus attached to a first end of the tube component, wherein the clamp adapter component comprises a clamp attachment part configured to attach the clamp adapter to a spinal clamp; providing an end assembly of the apparatus attached to a second end of the tube component, wherein the end assembly comprises a tension adjustment part configured to adjust a tension of the cable; and providing a sheath component of the apparatus surrounding a portion of the tube component, wherein the sheath component comprises a position locking mechanism configured to attach the sheath component to a base component.

### BRIEF DESCRIPTION OF THE DRAWINGS

FIG. 1 shows a planer view of an example of system for securing medical components according to aspects of the present disclosure.

FIG. 2 shows a side view of an example of system for securing medical components according to aspects of the present disclosure.

FIGS. 3 and 4 show examples of a medical apparatus according to aspects of the present disclosure.

FIG. 5 shows an example of a clamp adapter component according to aspects of the present disclosure.

FIG. 6 shows an example of an interlocking cylinder according to aspects of the present disclosure.

FIG. 7 shows an example of a sheath component according to aspects of the present disclosure.

FIG. 8 shows an example of a medical apparatus according to aspects of the present disclosure.

FIG. 9 shows an example of an end assembly according to aspects of the present disclosure.

FIG. 10 shows an example of a shell component according to aspects of the present disclosure.

FIG. 11 shows an example of a locking rod according to aspects of the present disclosure.

FIG. 12 shows an example of a tension adjustment part according to aspects of the present disclosure.

FIG. 13 shows an example of a cable according to aspects of the present disclosure.

FIGS. 14 and 15 show examples of methods for medical procedures according to aspects of the present disclosure.

### DETAILED DESCRIPTION

The present disclosure relates to medical devices. More specifically, the present disclosure may provide techniques and apparatuses that may be used to stabilize and support a patient during medical procedures.

Medical procedures may depend on stabilization of a patient and organization of a surgical environment, as well as routine sterilization of a surgical environment (e.g., of medical instruments, medical tables, and various other medical equipment). If a patient moves during a medical procedure, the procedure may be compromised, and elements of the environment may become unsterile. Therefore, there is a need in the art for systems and methods of stabilizing patients during medical procedures. In particular, there is a need for stabilizing a spine of a patient during procedures such as spinal screw placement.

Embodiments of the present disclosure provide systems and methods for stabilizing a patient during a medical procedure. For example, embodiments may be used to secure a spinous process clamp to a secure base. A surgical device according to the disclosure is designed to attach to a posterior element of the spine through process clamp and provide stability while minimizing unwanted spine motion during procedures such as a pedicle screw placement. It should be noted that the systems and methods described herein are not limited to these particular procedures and may be applied generally to stabilize a patient during any medical procedure that benefits from such stabilization.

Techniques and apparatuses described herein may also provide for improved organization and securement of medical components (e.g., such as cables, tubes, cords, medical instruments, medical drapery, etc.). For instance, embodiments of the present disclosure may be implemented to stabilize medical components (e.g., such as surgical devices, spinous process clamps, etc.), manage various cables and tubes, maintain sterilization of medical instruments and a surgical environment, stabilize a patient, etc. A channel component (e.g., a base, such as a modular base attachable to a platform) may include channels such that cords and tubes may be inserted through the channels and efficiently organized/managed by medical professionals.

As described in more detail herein, an apparatus may include a tube component, a cable (e.g., a cable running through the tube component), an end assembly (e.g., for adjusting tension of the cable), a clamp adapter component (e.g., to attach the cable/tube component to another medical device, such as a spinous process clamp), and a sheath component. The sheath component may be secured over tube components, and the sheath component may attach to a channel of a channel component (e.g., to secure the tube components to a base or a platform). Accordingly, tube components and cables (as well as other medical tubes, cords, etc.) may be secured in place via the channels to prevent any dislodging or unwanted movement. As such, medical equipment (e.g., surgical components, cables, etc.) may be secured, tube components and cords may flow to the patient in an organized fashion, etc.

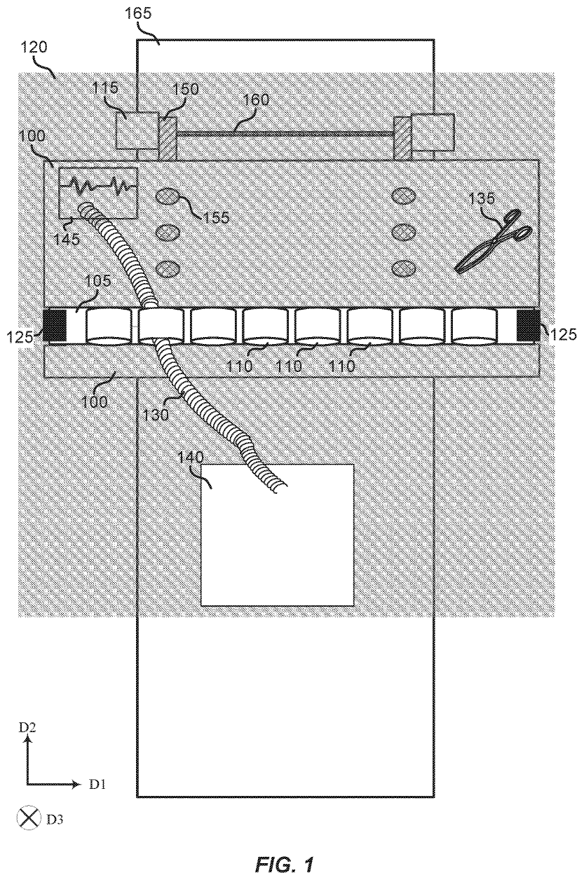

FIG. 1 shows an example of system for securing medical components according to aspects of the present disclosure. The example shown includes platform 100, channel component 105, channels 110, frame 115, medical drapery 120, attachment pieces 125, tube components 130, tools 135, patient area 140, a medical machine 145, support arms 150, attachment holes 155, support rod 160, and medical table 165.

According to some embodiments, platform 100 may refer to a platform (e.g., a medical table, a surgical table, a surgical tray, etc.) with a flat surface. In some examples, the platform 100 includes one or more notches (e.g., notches 420, notches 515), and the channel component 105 may be attached to the platform 100 via attachment pieces 125 aligned with the notches with the medical drapery 120 therebetween. In some examples, the platform 100 includes one or more peg holes, and the channel component 105 includes one or more pegs (e.g., pegs 810) corresponding to the one or more peg holes, where the peg holes are configured to receive the pegs with the medical drapery 120 therebetween.

According to some embodiments, platform 100 may refer to a platform (e.g., a medical table, a surgical table, a surgical tray, etc.) with a flat surface configured to support a medical drapery 120. In some examples, the platform 100 is configured to be movable in a third direction (D3) while the attached channel component 105 secures the at least one tube component 130 during the medical procedure, the third direction (D3) being substantially perpendicular to a plane formed by the first direction (D1) and the second direction (D2). In some examples, the platform 100 is configured to support one or more surgical tools 135 in addition to the attached channel component 105.

According to some embodiments, channel component 105 comprises one or more channels 110, wherein each of the channels 110 is configured to admit and secure one or more tube components 130 during a medical procedure. As described herein, the channel component 105 may be configured to attach to the platform 100 and secure a medical drapery 120 therebetween (e.g., via attachment pieces 125 and notches in the platform 100 and channel component 105, via peg holes in the platform and pegs of the channel component 105, or via various other mechanisms for attaching the channel component 105 to the platform 100). In some examples, each of the channels 110 includes a portion of a cylinder with an opening configured to receive the one or more tube components 130.

In some examples, a tube component 130 may include an electrical cord, a bipolar cord, a suction tube, a water tube, a drill cord, a smoke evacuation cord, an electrocautery cord, an electrical stimulation cord, various other cords, or any combination thereof. In some examples, a tube component 130 may include cables, an IV drip, feeding tubes, inhalational anesthetic tubes, various other tubes, or any combination thereof. In some aspects, the terms “cord,” “tube,” etc. may be used interchangeably to refer to any components (e.g., medical components) securable by a channel component 105.

According to some embodiments, channel component 105 comprises a plurality of channels 110, wherein the channel component 105 is substantially the same size as the platform 100 in a first direction (D1) and is configured to attach to the platform 100 to secure the medical drapery 120 during a medical procedure. In some examples, the plurality of channels 110 is arranged in the first direction (D1) and is configured to admit and secure at least one tube component 130 during the medical procedure. In some examples, each of the channels 110 in the set of channels 110 includes a portion of a cylinder with an opening configured to receive one or more tube components 130, the one or more tube components 130 extending from the channel component 105 along a second direction (D2) substantially perpendicular to the first direction. In some examples, the channel component 105, the channels 110, and any channel locks (e.g., channel locks 215, which may include or refer to a sheath component) are sterilizable.

Channel component 105 may include one or more channels 110, and various channels 110 or combinations of channels 110 may further include a channel lock. A channel 110 may include, or refer to, a base component. In some aspects, a channel 110 may include, or refer to, a base component and a top component. In some aspects, a channel 110 may further include, or refer to, a base component, a top component, and a sheath component.

According to some embodiments, attachment pieces 125 may include a top extension and a bottom extension and may be configured to secure one or more ends of the channel component 105 to one or more sides of the platform 100. In some examples, each of the one or more sides of the platform 100 comprises a groove (e.g., a notch) configured to receive the one or more attachment pieces 125, and the channel component 105 comprises an attachment notch configured to receive the top extension. In some examples, the bottom extension includes an extension peg, and the platform 100 includes an extension peg hole configured to receive the extension peg.

Attachment pieces 125 may generally include or refer to any attachment devices, clamps, hinged clamps, grips, brackets, fasteners, vices, etc. Tools 135 may generally include or refer to medical components such as scalpels, forceps, needles/syringes, spreaders, clamps, saws, rods, screws, or any other medical equipment.

In some cases, patient area 140 may refer to an area or region of a patient undergoing a medical procedure (e.g., such as a surgery). In some cases, patient area 140 may refer to an area set up by medical professionals as an area in which a surgical procedure may be performed. For instance, in some cases a patient area 140 may be formed such that a sterile environment surrounds the patient area 140 (e.g., where medical drapery 120 may surround the patient area 140, nearby medical components may be sterilized above medical drapery 120, medical components used for the medical procedure may be sterilized, etc.). Medical machines 145 may include or refer to any medical device or machine used for medical procedures. For instance, examples of a medical machine 145 may include an IV drip bag, a feeding machine, a pulse monitor, an anesthesia machine, etc.

According to some embodiments, support arms 150 are configured to attach the platform 100 to a frame 115 (e.g., a surgical bed frame). In some examples, the one or more support arms 150 may be permanently attached to the platform 100, and the channel component 105 may be separable from the platform 100.

According to some embodiments, support rods 160 may be configured to be inserted through first holes in the frame 115 (e.g., a surgical bed frame) and through second holes in the one or more support arms 150, thereby securing the apparatus to the frame 115. In some examples, a patient undergoing a medical procedure (e.g., surgery) may be positioned on a medical table 165. In some cases, medical table 165 may include or refer to a surgical bed. In some cases, frame 115 and medical table 165 may be a single component (e.g., a surgical bed frame, which may support a patient via a flat surface spanning a first and second direction, D1 and D2, and supply a mounting frame 115 in a third direction, D3)).

As further described herein, channel component 105 may be attached to the platform 100 in order to efficiently secure medical drapery 120 and facilitate sterilization of appropriate medical components (e.g., in addition to providing for improved organization of medical equipment, such as any of various tube components 130 and tools 135 that may be used in a medical procedure). For instance, elements under medical drapery 120 (e.g., platform 100, various parts of frame 115 such as support arms 150, support rods 160, etc.). Alternatively, elements above medical drapery 120 (e.g., and in proximity of patient area 140) may be sterile or sterilized prior to, or during, a medical procedure. For instance, one or more medical machines 145 may be under medical drapery 120 (e.g., on platform 100 in a direction D2 away from the patient area, on the floor, etc.) and portions of one or more tube components 130 attached to each of the medical machines 145 may be passed around the medical drapery 120 and through a channel 110 of the channel component 105. Accordingly, tube components 130 may be sterilized and medical drapery 120 may be sterilized or disposed of after medical procedures without requiring the platform 100, the various medical machines 145, etc. be sterilized (e.g., as the medical drapery 120 may provide a barrier between non-sterilized elements or medical components and the patient area 140).

According to some embodiments, channel component 105 may include channels 110 (e.g., each channel 110 including a portion of cylinder with an opening configured to potentially receive a tube component 130) and channel locks (e.g., each channel lock configured to attach to one of the channels 110, thereby confining any tube components 130 to the corresponding channel 110) which may all be readily sterilized. That is, channel component 105 (e.g., and all of channel component 105 elements or subcomponents) may be separable from platform 100 and may be sterilized between medical procedures (e.g., along with other medical components such as tools 135. Accordingly, the techniques and apparatuses described herein may provide for efficient securement of medical drapery 120, improved organization of tube components 130 and other tools 135, and may facilitate medical procedures (e.g., via improved medical professional efficiency, surgical environment optimization, etc.) while satisfying sterilization demands

In some cases, channel component 105 (e.g., and any elements, subcomponents, or pieces of a channel component 105) may be disposable, may be sterilized with other medical instruments (e.g., with any tools 135 used in a procedure), may be sterilized and pre-packaged or included in kits of surgical instruments, may be sterilized via sterilization trays or other sterilization receptacles, etc. Accordingly, channel component 105 may be suitable for securing medical drapery 120, for organizing medical components (e.g., such as tube components 130), etc.

Further, according to some embodiments, a sterile channel component 105 may provide for secure attachment to platform 100 (e.g., and thus to a rigid or sturdy frame 115 and/or medical table 165). As such, sterile channel component 105 may be utilized to stabilize a patient or one or more aspects of patient area 140 (e.g., in addition to one or more tube components 130 or any other medical components). For instance, sterile channel component 105 may be suitable for stabilizing a patient's spine for surgical procedures, in addition to stabilizing one or more tubes or tube components 130 for performing the surgical procedures.

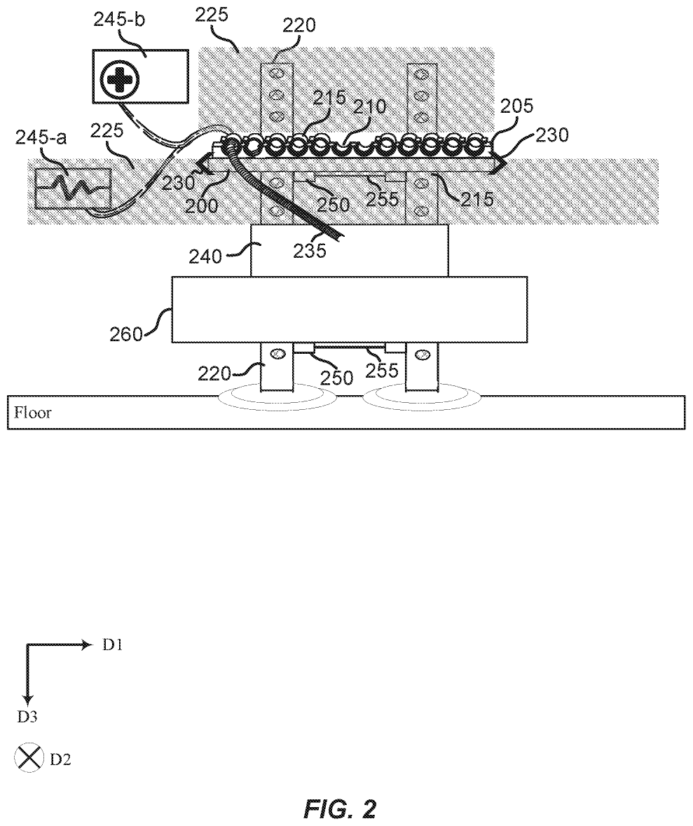

Platform 100, channel component 105, channels 110, frame 115, medical drapery 120, attachment pieces 125, patient area 140, medical machines 145, support arms 150, support rods 160, and medical table 165 are each examples of, or each include aspects of, the corresponding elements described with reference to FIG. 2. Tube components 130 are examples of, or include aspects of, the corresponding elements described with reference to FIGS. 2-4.

FIG. 2 shows an example of a system for securing medical components according to aspects of the present disclosure. The example shown includes platform 200, channel component 205, channels 210, channel locks 215, frame 220, medical drapery 225, attachment pieces 230, tube components 235, patient area 240, medical machines 245, support arms 250, support rods 255, and medical table 260.

A channel component 205 (e.g., which may be referred to as a base, a Rotolock Modular Base, etc.) may include channels 210 (e.g., a sequence or row of channels 210 formed by base components and top components), such that cords and tubes may be inserted through the channels and efficiently organized/managed by medical professionals. For instance, an apparatus or system described herein may include a base component, a top component, and a sheath component. The sheath component may be secured over tube components 235 (e.g., over an interlocking cylinder of a tube component 235, as described in more detail herein) of varying sizes, and the sheath component may then be secured to the base component. In some examples, multiple base components may be connected together, providing possible configuration of multiple channels when combined with additional top components/sheath components. Accordingly, various tube components 235 may be secured in place via the channels 210 to prevent tube components 235 from becoming dislodged, to direct tube components 235 to a patient area 240 or to one or more medical machines 245 in an organized fashion, to stabilize tube components 235 relative to a medical table 260, to stabilize tube components 235 relative to a patient area 240, etc.

In some examples, a patient may be positioned on a medical table 260. A patient area 240 may then be formed or established for performing a medical procedure (e.g., such as a surgical procedure) on the patient. For example, after the patient is positioned on the medical table 260, a platform 200 may be attached near the patient area 240 and medical drapery 225 may be placed over the platform 200. In some cases, the platform 200 may be attached to support arms 250 that may secure the platform 200 to a frame 220 (e.g., via two support rods 200) going through holes in the support arms 250 and the frame 220. Channel component 205 may then be attached to platform 200 to secure medical drapery 225 therebetween (e.g., between the platform 200 and the channel component 205). For instance, channel component 205 may be attached to platform 200 via attachment pieces 230.

As described herein, channel component 205 may include a plurality of channels 210 (e.g., such as parallel channels 210 that may be arranged in sequence along a first direction, D1). Further, channel component 205 may include channel locks 215 corresponding to any number of the plurality of channels 210 (e.g., channel component 205 may include zero channel locks 215, channel locks 215 for each channel 210 of a subset of the plurality of channels 210, or channel locks 215 for each channel 210 of all channels of the plurality of channels 210). As described, the channels 210 each include a portion of a cylinder with an opening configured to receive one or more tube components 235 and the channel locks 215 each include a sliding portion (e.g., a position locking mechanism configured to attach channel locks 215 to the channel component or the base component). Further, each of the channels 210 (e.g., each base component) may include a groove configured to receive the sliding portion, and each of the channel locks 215 further include a notch configured to secure the channel lock 215 in a closed position. Accordingly, one or more tube components 235 may be secured by each channel 210 and corresponding channel lock 215 without pinching, impeding, or damaging any of the secured tube components 235. As an example, for some medical procedure applications, each channel 210 and corresponding channel lock 215 combination (e.g., when closed, when locked, when in a tube component 235 securing position, etc.) may have a diameter of 0.75 inches.

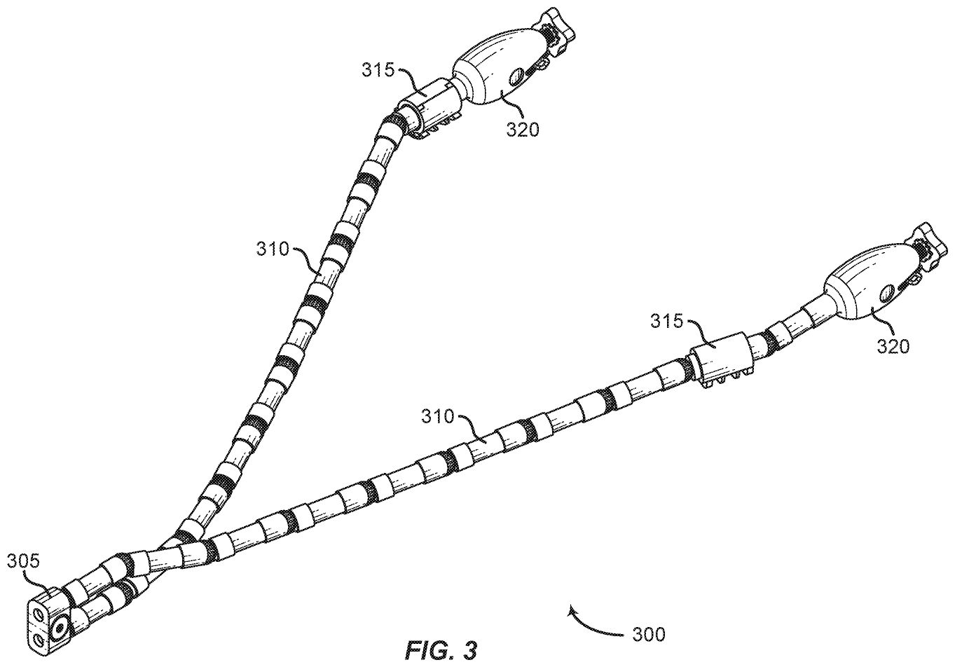

According to some embodiments, channel locks 215 may be configured to attach to one of the channels 210, thereby confining the tube component 235 to the corresponding channel. In some examples, each of the channel locks 215 includes a sliding portion, and each of the channels 210 includes a groove configured to receive the sliding portion, and where each of the channel locks 215 further includes a notch configured to secure the channel lock in a closed position. According to some embodiments, channel locks 215 may be configured to attach to at least one channel of the plurality of channels 210, thereby confining one or more tube components 235 to the corresponding channel. In some examples, channel locks 215 may include or refer to sheath components described herein (e.g., with reference to FIGS. 3, 4, and 7).

In some example, frame 200 and medical table 260 may be a single component (e.g., such as a surgical bed frame 220). In other examples, frame 200 may be a separate component from medical table 260, and the frame 220 may attach to the medical table 260 via support arms 250 attached to the medical table 260 (e.g., where one or more support rods 255 may attach the medical table 260 and the frame 220 through holes in each of the medical table 260 and the frame 220).

Various platforms 200 for securing medical components may be implemented in accordance with the described techniques without departing from the scope of the present disclosure. For instance, a platform 200 may be designed and implemented based on various factors such as the surgical environment (e.g., the size of the operating room), the medical table 260 and/or the frame 220 (e.g., the type or dimensions of the medical table 260 and/or the frame 220), the medical components being used (e.g., the types of medical instruments or the amount of medical instruments), etc. As an example, a platform 200 include a platform of 12 inches in a second direction D2 (e.g., in length) and 18 inches in a first direction D1 (e.g., in width), with various heights (e.g., with various dimensions in a third direction D3). As another example, a platform 200 include a platform of 24 inches in a second direction D2 (e.g., in length) and 18 inches in a first direction D1 (e.g., in width), with various heights (e.g., with various dimensions in a third direction D3). In some cases, the platform 200 may attach directly to (e.g., and have dimensions facilitating direct attachment to) the frame 220 used in the surgical setting (e.g., such as a Jackson frame).

In some cases, platform 200 may attach to frame 220 (e.g., which in some cases may refer to a metal piece with all the holes in it, such as an “H” bracket). For instance, frame 220 (e.g., a H bracket) may have 1 hole to accept a small post in the vertical direction, and a second hole to slide the metal cylinder pin through. Brackets of other platforms 200 (e.g., platforms 200 of varying dimensions) may be designed to fit on the bed (e.g., medical table 260) the same way the H bracket is designed to fit on, accept the brackets may travel horizontally instead or vertically. In some cases, the platform may use a single pin to attach to the bed.

Platform 200, channel component 205, channels 210, frame 220, medical drapery 225, attachment pieces 230, patient area 240, medical machines 245, support arms 250, support rods 255, and medical table 260 are each examples of, or each include aspects of, the corresponding elements described with reference to FIG. 1. Tube components 235 are examples of, or include aspects of, the corresponding elements described with reference to FIGS. 1, 3, and 4.

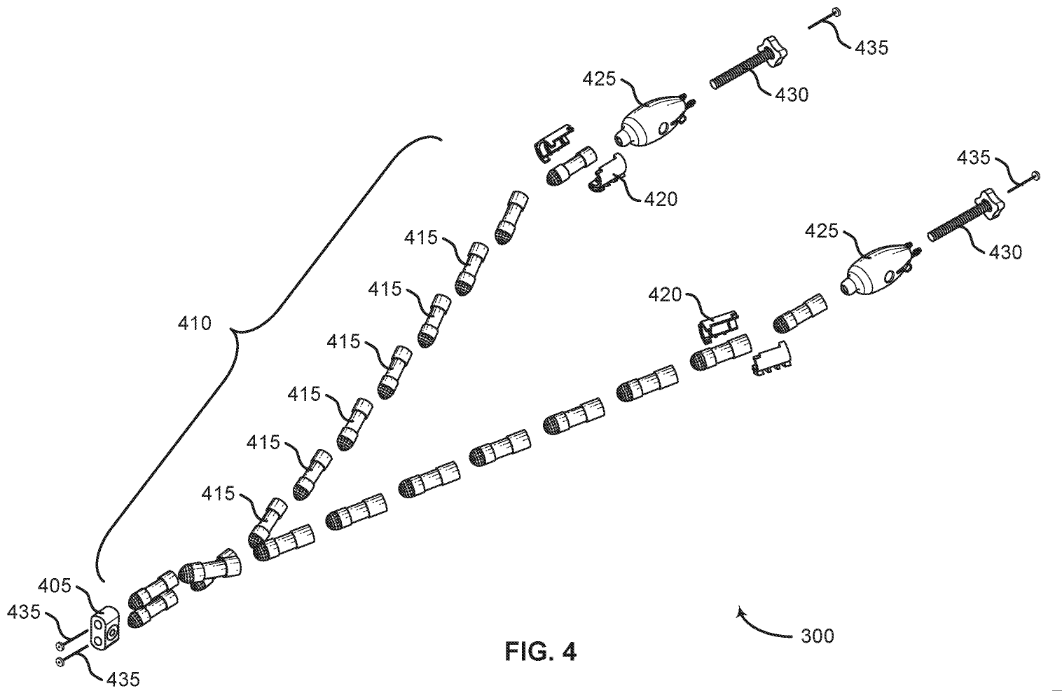

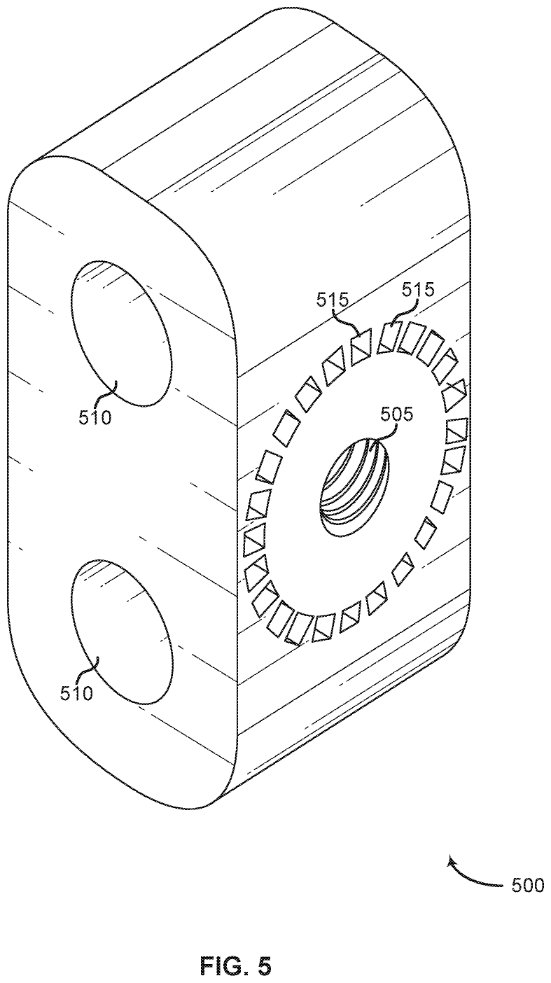

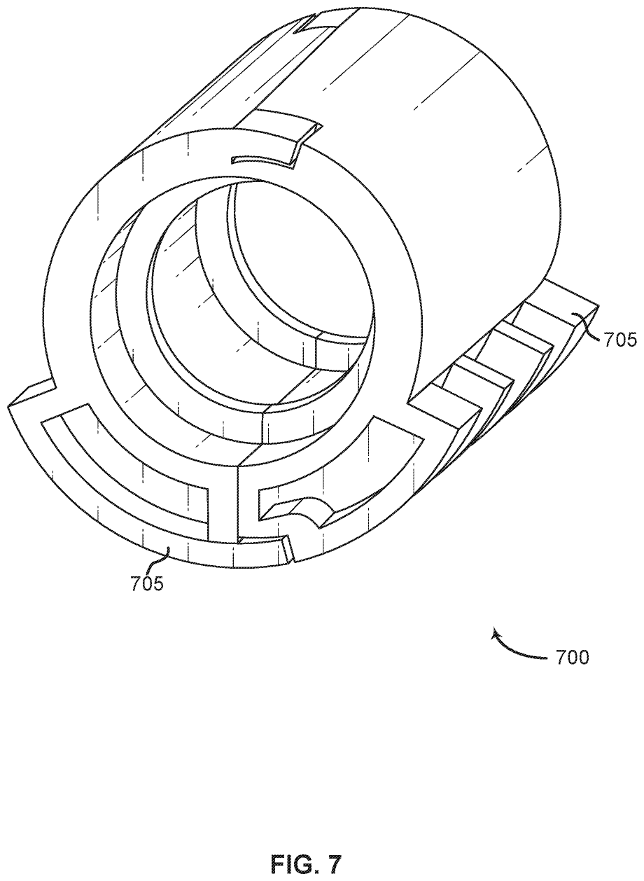

FIG. 3 shows an example of a medical apparatus 300 according to aspects of the present disclosure. Medical apparatus 300 is an example of, or includes aspects of, the corresponding element described with reference to FIG. 4. In one aspect, medical apparatus 300 includes clamp adapter component 305, tube component 310, sheath component 315, and end assembly 320. Clamp adapter component 305 is an example of, or includes aspects of, the corresponding element described with reference to FIGS. 4 and 5. Tube component 310 is an example of, or includes aspects of, the corresponding element described with reference to FIGS. 1, 2, and 4. Sheath component 315 is an example of, or includes aspects of, the corresponding element described with reference to FIGS. 4 and 7. End assembly 320 is an example of, or includes aspects of, the corresponding element described with reference to FIGS. 4, 8, and 9.

An example medical apparatus 300 (e.g., a surgical apparatus, a spine stabilization apparatus, etc.) is described. In some embodiments, the medical apparatus 300 may include a clamp adapter component 305, one or more tube components 310, one or more cables, one or more end assemblies, and one or more sheath components 315. In some aspects, each of the one or more cables extend through a corresponding tube component 310. The clamp adapter component 305 may be attached to a first end of each of the one or more tube components 310 (e.g., where the clamp adapter component 305 comprises a clamp attachment part). The one or more end assemblies may be attached to a second end of each of the one or more tube components 310, respectively. In some aspects, each of the end assemblies include a tension adjustment part configured to adjust a tension of a corresponding cable. Further, the one or more sheath components 315 may surround a portion of each of the one or more tube components 310, respectively, where each of the sheath components 315 include a position locking mechanism (e.g., locking the sheath component 315 and tube component 310 to a base component, for example).

In some aspects, a spinal clamp may be attached to the clamp adapter component 305 (e.g., where the spinal clamp may be configured to attach to a spine of a patient during a medical procedure). In some examples, a base component may be attached to each of the plurality of sheath components 315 using the position locking mechanism (e.g., where the base component may be attached to a fixed surface). In some examples, the base component may include a channel component described with reference to FIGS. 1 and 2.

The medical apparatuses described herein may be used for various medical procedures, surgeries, operations, etc. For instance, in some implementations, the medical apparatus 300 may be attached to the posterior elements of a patients spine through a spinous process clamp, and the medical apparatus 300 may provide stability while minimizing unwanted spine motion during robotics-assisted pedicle screws placement.

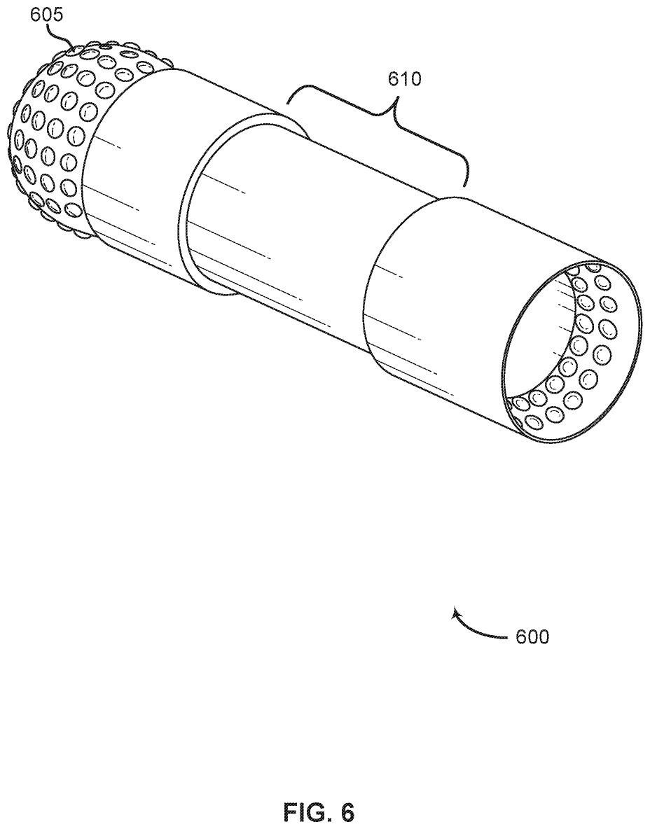

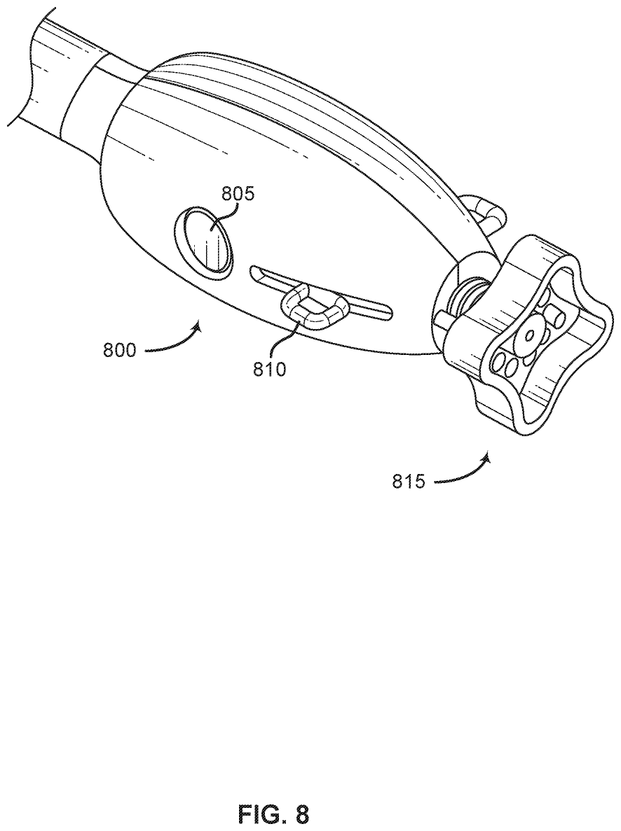

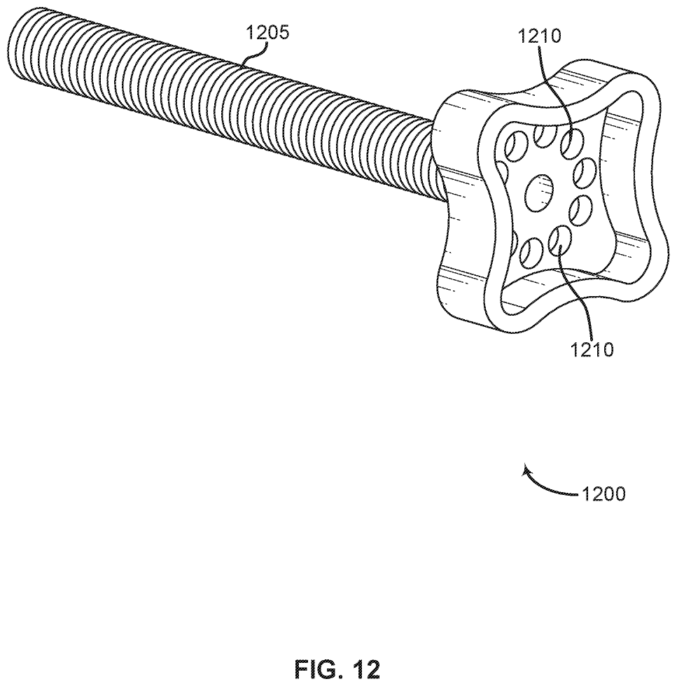

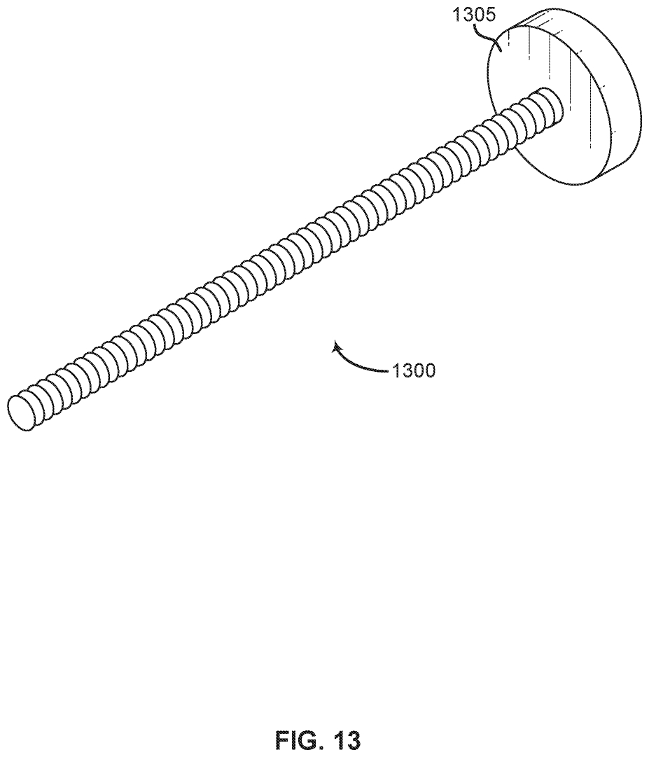

FIG. 4 shows an example of a medical apparatus 400 (e.g., an assembly view of a medical apparatus 400) according to aspects of the present disclosure. Medical apparatus 400 is an example of, or includes aspects of, the corresponding element described with reference to FIG. 3. In one aspect, medical apparatus 400 includes clamp adapter component 405, tube component 410, sheath component 420, end assembly 425, tension adjustment part 430, and cable 435. Clamp adapter component 405 is an example of, or includes aspects of, the corresponding element described with reference to FIGS. 3 and 5. Tube component 410 is an example of, or includes aspects of, the corresponding element described with reference to FIGS. 1-3. In one aspect, tube component 410 includes interlocking cylinders 415 (e.g., in the example of FIG. 4, each of the two tube components 410 include ten interlocking cylinders 415, although any number of interlocking cylinders 410 and any number of tube components 410 may be implemented by analogy, without departing from the scope of the present disclosure). Interlocking cylinder 415 is an example of, or includes aspects of, the corresponding element described with reference to FIG. 6. Sheath component 420 is an example of, or includes aspects of, the corresponding element described with reference to FIGS. 3 and 7. End assembly 425 is an example of, or includes aspects of, the corresponding element described with reference to FIGS. 3, 8, and 9. Tension adjustment part 430 is an example of, or includes aspects of, the corresponding element described with reference to FIGS. 8 and 12. Cable 435 is an example of, or includes aspects of, the corresponding element described with reference to FIG. 13.

A medical apparatus 400 (e.g., a surgical apparatus, a spine stabilization apparatus, etc.) is described. In some embodiments, the medical apparatus 400 may include at least a clamp adapter component 405, a tube component 410, a sheath component 420, an end assembly 425 and a cable 435 (e.g., where the cable 435 extends through the tube component 410). In some aspects, the clamp adapter component 405 is attached to a first end of the tube component 410. The clamp adapter component 405 may include a clamp attachment part configured to attach the clamp adapter to a spinal clamp. The end assembly 425 may be attached to a second end of the tube component 410. The end assembly 425 may include a tension adjustment part 430 configured to adjust a tension of the cable 435. Further, the sheath component 420 may surround a portion of the tube component 410, for example, where the sheath component 420 includes a position locking mechanism configured to attach the sheath component 420 to a base component.

In some embodiments, the medical apparatus 400 may include additional components. For instance, in the example of FIGS. 3 and 4, the medical apparatus 400 may include an additional tube component 410 attached to the clamp adapter component 405, an additional cable 435 extending through the additional tube component 410, and an additional end assembly 425 attached to the additional tube component 410. The cable 435 extends through a first hole in the a clamp adapter component 405, and the additional cable 435 extends through a second hole in the clamp adapter component 405 (e.g., aspects of which are described in more detail herein, for example, with reference to FIG. 5).

In some aspects (e.g., as described in more detail herein, for example, with reference to FIG. 6), each tube component 410 may include multiple interlocking cylinders 415 (e.g., where the interlocking cylinders 415 each include a first end configured to attach to a second end of a subsequent interlocking cylinder 415).

In some aspects, the tension adjustment part 430 comprises a threaded rod configured to adjust a tension of the cable 435 within the tube component 410 when the threaded rod is rotated. For instance, the end of each cable 435 may include a retaining disk to secure the cable 435 against the tension adjustment part 430. As the threaded rod of the tension adjustment part 430 is rotated, the tension of the cord may be adjusted as the length of the threaded rod is increased or decreased relative to the end assembly 425. When the tension of the cord or cable 435 is adjusted using the tension adjustment part 430, the device (e.g., the interlocking cylinders 415) may become more rigid due to compression applied by the ends of the cable 435. Thus, the compression on the apparatus may cause the apparatus to become more rigid, which may then stabilize a position of a part of a patient's body attached to the apparatus (e.g., stabilizing the spine via a spinal clamp).

FIG. 5 shows an example of a clamp adapter component 500 according to aspects of the present disclosure. Clamp adapter component 500 is an example of, or includes aspects of, the corresponding element described with reference to FIGS. 3 and 4. In one aspect, clamp adapter component 500 includes clamp attachment part 505, hole 510, and teeth indentations 515.

Each cable may extend through a hole 510 in the clamp adapter component 500. For instance, a first cable may extend through a first hole 510 in the clamp adapter component 500 and an additional cable (e.g., a second cable) may extend through a second hole 510 in the clamp adapter component 500. The clamp attachment part 505 may include a threaded screw hole 510 (e.g., for attaching the clamp attachment part 505 to another medical component, such as to a spinal clamp). Moreover, in some examples, the clamp attachment part 505 includes a plurality of teeth indentations 515, which may be configured to adjustably position another medical component (e.g., a spinal clamp) with respect to the clamp attachment part 505.

In some examples, a clamp adapter component 500 (e.g., which in some cases may be referred to as a snake clamp adapter component 500) may attach a snake assembly (e.g., tube components, interior cables, etc.) to a medical component, for example, such as a spinous process clamp. For example, the clamp adapter component 500 may attach to a spinous process clamp with a threaded screw hole 510. The clamp adapter component 500 may change attachment angles using teeth indentions on the side. In some examples, two cables may be attached to the spinous process clamp through the thru-holes 510 and the cables travel through the device to a handle assembly to provide improved stability. In some examples, a dimpled texture is present on back of the thru-holes 510 to provide resistance to motion.

FIG. 6 shows an example of an interlocking cylinder 600 according to aspects of the present disclosure. Interlocking cylinder 600 is an example of, or includes aspects of, the corresponding element described with reference to FIG. 4. In one aspect, interlocking cylinder 600 includes dimples 605 and recess 610.

As described herein, each tube component may include one or more interlocking cylinders 600. In some examples, each interlocking cylinder 600 may include a first end configured to attach to a second end of a subsequent interlocking cylinder 600.

The tube component (e.g., and thus each interlocking cylinder 600 of the tube component) is hollow, allowing for a cable to travel through the tube component). In some cases, ten or more interlocking cylinders 600 may be connected to provide a flexible structure (e.g., a flexible tube component) up to a channel component (e.g., a Rotolock base) and platform system. The dimples 605 in the dome area of each interlocking cylinder 600 may provide texture and rigidity once a position is maintained. Moreover, the recess 610 on the outer body of each interlocking cylinder 600 allows for the sheath to attach and secure the device to the channel component (e.g., a Rotolock base), which allowing for highly configurable attachment and positioning, as each interlocking cylinder 600 may be selected for sheath component attachment.

FIG. 7 shows an example of a sheath component 700 according to aspects of the present disclosure. Sheath component 700 is an example of, or includes aspects of, the corresponding element described with reference to FIGS. 3 and 4. In one aspect, sheath component 700 includes position locking mechanism 705.

The position locking mechanism 705 may include a curved extension configured to be inserted into a curved space within a base component (e.g., within a channel component or a Rotolock Modular Base described herein, for example, with reference to FIGS. 1 and 2).

In one example, the sheath component 700 may attach to an interlocking cylinder (e.g., to any interlocking cylinder of the tube component) using two pieces surrounding the interlocking cylinder, and the sheath component 700 may clamp together surrounding the selected interlocking cylinder. The sheath component 700 may allow the interlocking cylinder, and therefore the tube component, to attach to a channel component, where the channel component may be attached to a platform (e.g., an operating room bed) for stability. The shape of the sheath component 700 may be configured to (e.g., compatible to) slide into a channel component (e.g., Rotolock top) and together rotate into the base. The inner struts of the sheath component 700 may fit into, and be secured to, a recess on an interlocking cylinder for stability.

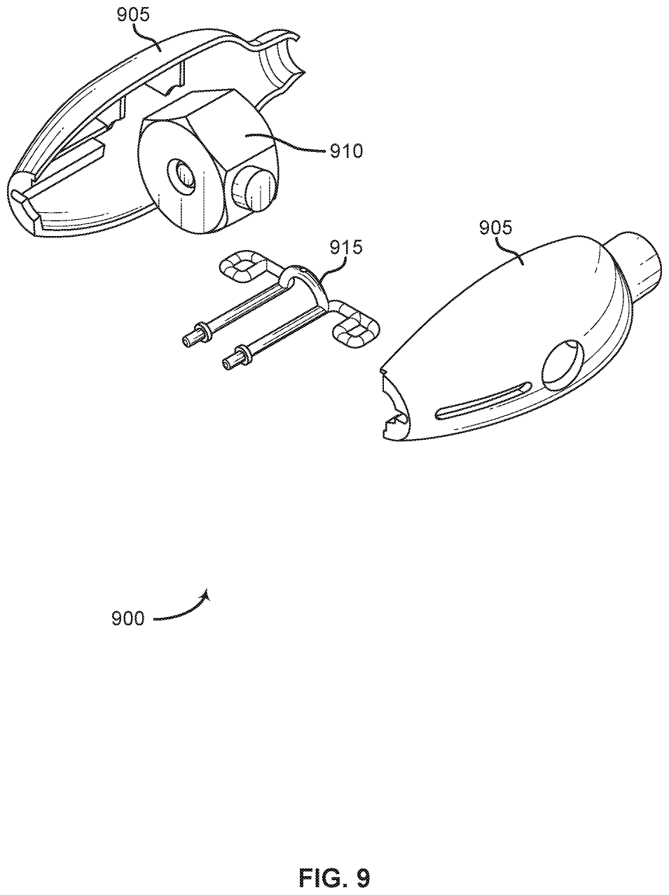

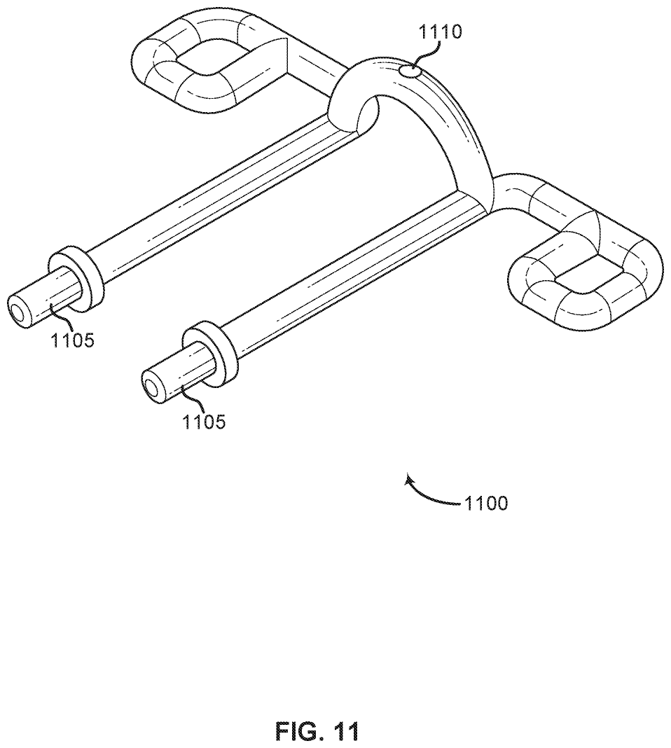

FIG. 8 shows an example of a medical apparatus according to aspects of the present disclosure. The example shown includes end assembly 800 and tension adjustment part 815. End assembly 800 is an example of, or includes aspects of, the corresponding element described with reference to FIGS. 3, 4, and 9. In one aspect, end assembly 800 includes quick release nut 805 and locking rod 810. Quick release nut 805 is an example of, or includes aspects of, the corresponding element described with reference to FIG. 9. Locking rod 810 is an example of, or includes aspects of, the corresponding element described with reference to FIGS. 9 and 11. Tension adjustment part 815 is an example of, or includes aspects of, the corresponding element described with reference to FIGS. 4 and 12.

The end assembly 800 may include a shell component (e.g., where the shell component may include two interlocking halves). The end assembly 800 may include a quick release nut 805 and a spring-loaded locking rod 810 (e.g., as described in more detail herein, for example, with reference to FIGS. 9-11).

A end assembly 800 (e.g., which may be referred to as a snake handle assembly) may provide a tension and locking mechanism when the end assembly 800 is positioned in a desired location. A button (e.g., a quick release nut 805 button) on a side is configured to be a quick release or large adjustment tensioner. A tension adjustment part 815 (e.g., a rotating knob) on the back of the end assembly 800 is a fine adjustment tensioner mechanism (e.g., a threaded rod described in more detail herein, for example, with reference to FIG. 12). A locking mechanism (e.g., locking rod 810) may be a spring-loaded slide used to secure the rotating knob in the tensioned position.

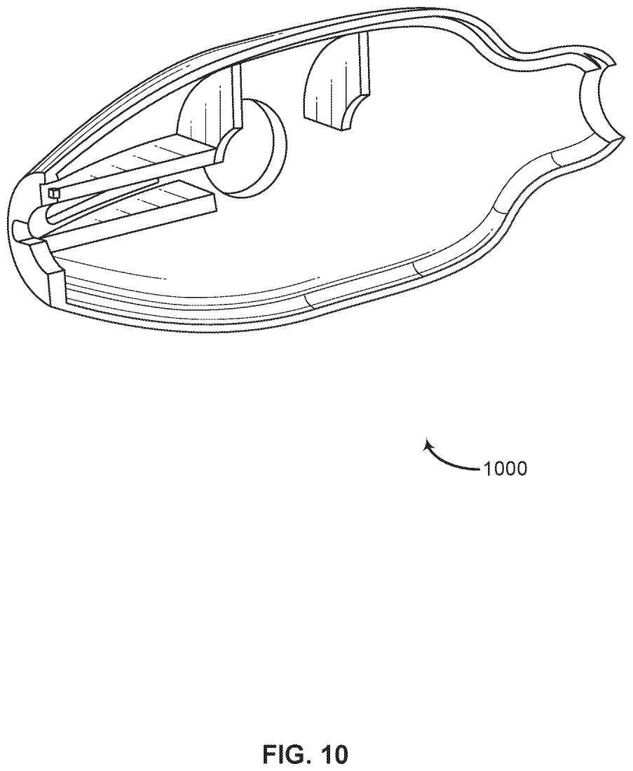

FIG. 9 shows an example of an end assembly 900 according to aspects of the present disclosure. End assembly 900 is an example of, or includes aspects of, the corresponding element described with reference to FIGS. 3, 4, and 8. In one aspect, end assembly 900 includes shell component 905, quick release nut 910, and locking rod 915. Shell component 905 is an example of, or includes aspects of, the corresponding element described with reference to FIG. 10. Quick release nut 910 is an example of, or includes aspects of, the corresponding element described with reference to FIG. 8. Locking rod 915 is an example of, or includes aspects of, the corresponding element described with reference to FIGS. 8 and 11.

In some aspects, the end assembly 900 may include a shell component 905 (e.g., outer shells), a quick release nut 910, a spring-loaded locking rod 915 (e.g., a locking rod 915), and a threaded knob. The shell component 905 (e.g., outer shells) may be bolted, screwed, glued, or adhered in any other manner (e.g., to form the end assembly 900).

FIG. 10 shows an example of a shell component 1000 according to aspects of the present disclosure. Shell component 1000 is an example of, or includes aspects of, the corresponding element described with reference to FIG. 9.

In some embodiments, the shell component 1000 may include a spring attachment and inner rails to provide stability for the locking rod. In some examples, the shell component 1000 may also include vertical rails to hold the quick release nut in position. In some embodiments, the outer edge of the shell component 1000 includes recessed snaps to hold the two outer shells together.

FIG. 11 shows an example of a locking rod 1100 according to aspects of the present disclosure. Locking rod 1100 is an example of, or includes aspects of, the corresponding element described with reference to FIGS. 8 and 9. In one aspect, locking rod 1100 includes rails 1105 and hole 1110.

In some examples, the locking rod 1100 may be shaped to fit within the assembled end assembly (e.g., within the shell component of the end assembly). The hole 1110 on the back arch may be included as a spring attachment. The rails 1105 on the two ends of the locking rod 1100 may lock into the tension adjustment part (e.g., into the face of a threaded knob) once ideal tension is achieved (e.g., aspects of which are shown in FIGS. 8 and 12).

FIG. 12 shows an example of a tension adjustment part 1200 according to aspects of the present disclosure. Tension adjustment part 1200 is an example of, or includes aspects of, the corresponding element described with reference to FIGS. 4 and 8. In one aspect, tension adjustment part 1200 includes threaded rod 1205 and holes 1210.

In some embodiments, the tension adjustment part 1200 includes a threaded rod 1205 configured to adjust a tension of the cable (e.g., when the threaded rod 1205 is rotated). The tension adjustment part 1200 may travel through the end assembly, and the tension adjustment part 1200 (e.g., the threaded rod 1205 of the tension adjustment part 1200) may be threaded into a quick release nut of the end assembly. The tension adjustment part 1200 may be hollow to allow a cable to travel through the tension adjustment part 1200 (e.g., through the knob) for cable tensioning. The holes 1210 in the tension adjustment part 1200 (e.g., in the face of the knob of the tension adjustment part 1200) may allow the locking rod (e.g., the rails of the locking rod) to seat into the knob to prevent any turning and releasing of the tension.

FIG. 13 shows an example of a cable 1300 according to aspects of the present disclosure. Cable 1300 is an example of, or includes aspects of, the corresponding element described with reference to FIG. 4. In one aspect, cable 1300 includes retaining disk 1305.

As described herein, a retaining disc may be included or configured on each end each cable 1300 (e.g., to secure the cable 1300 through the tube component on the ends of each of the clamp adapter component and the end assembly). The retaining disc attaches to the cable 1300 after full assembly to allow rigid tensioning. Both ends of the cable 1300 may include a retaining disc.

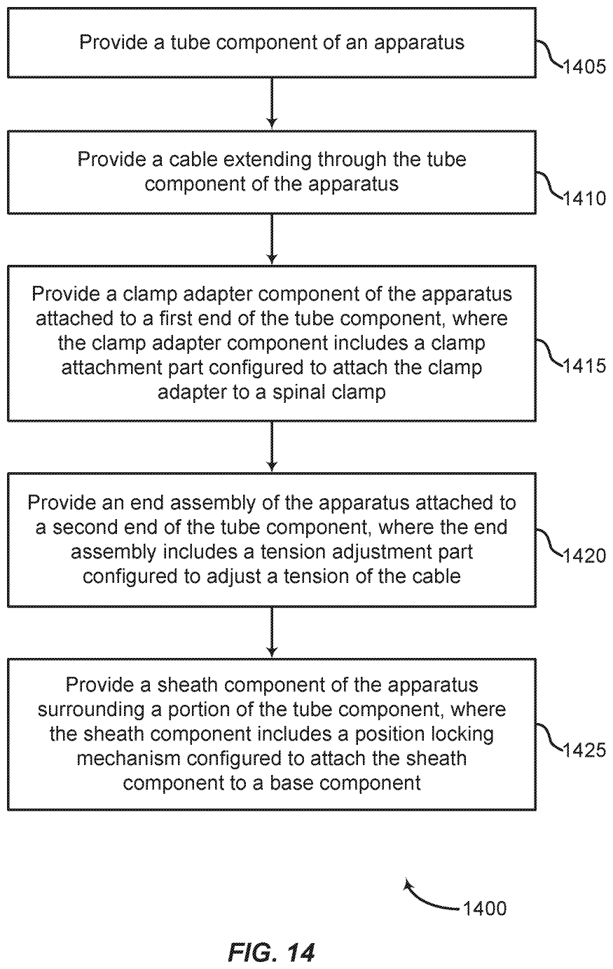

FIG. 14 shows an example of a method 1400 for medical procedures according to aspects of the present disclosure. In some examples, these operations are performed by a system including a processor executing a set of codes to control functional elements of an apparatus. Additionally or alternatively, certain processes are performed using special-purpose hardware. Generally, these operations are performed according to the methods and processes described in accordance with aspects of the present disclosure. In some cases, the operations described herein are composed of various substeps, or are performed in conjunction with other operations.

In some cases, various aspects of operations 1405-1425 may be performed by, or facilitated by a medical professional (e.g., as described in more detail herein).

At operation 1405, the system provides a tube component of an apparatus. In some cases, the operations of this step refer to, or may be performed by, a tube component as described with reference to FIGS. 1-4.

At operation 1410, the system provides a cable extending through the tube component of the apparatus. In some cases, the operations of this step refer to, or may be performed by, a cable as described with reference to FIGS. 4 and 13.

At operation 1415, the system provides a clamp adapter component of the apparatus attached to a first end of the tube component, where the clamp adapter component includes a clamp attachment part configured to attach the clamp adapter to a spinal clamp. In some cases, the operations of this step refer to, or may be performed by, a clamp adapter component as described with reference to FIGS. 3-5.

At operation 1420, the system provides an end assembly of the apparatus attached to a second end of the tube component, where the end assembly includes a tension adjustment part configured to adjust a tension of the cable. In some cases, the operations of this step refer to, or may be performed by, an end assembly as described with reference to FIGS. 3, 4, 8, and 9.

At operation 1425, the system provides a sheath component of the apparatus surrounding a portion of the tube component, where the sheath component includes a position locking mechanism configured to attach the sheath component to a base component. In some cases, the operations of this step refer to, or may be performed by, a sheath component as described with reference to FIGS. 3, 4, and 7.

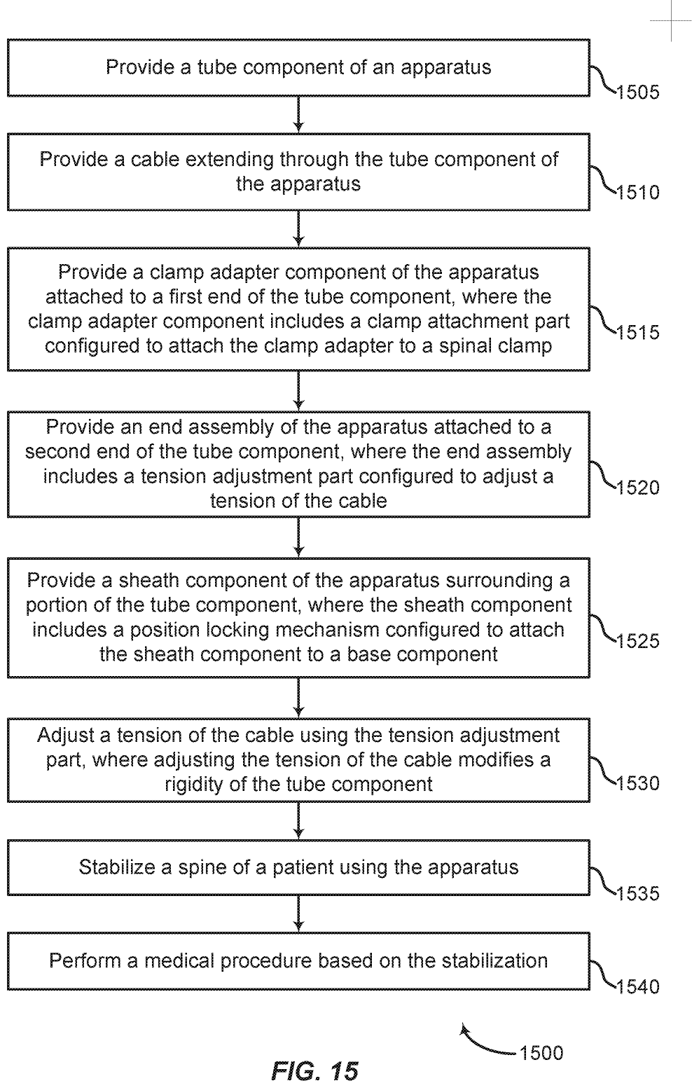

FIG. 15 shows an example of a method 1500 for medical procedures according to aspects of the present disclosure. In some examples, these operations are performed by a system including a processor executing a set of codes to control functional elements of an apparatus. Additionally or alternatively, certain processes are performed using special-purpose hardware. Generally, these operations are performed according to the methods and processes described in accordance with aspects of the present disclosure. In some cases, the operations described herein are composed of various substeps, or are performed in conjunction with other operations.

In some cases, various aspects of operations 1505-1540 may be performed by, or facilitated by a medical professional (e.g., as described in more detail herein).

At operation 1505, the system provides a tube component of an apparatus. In some cases, the operations of this step refer to, or may be performed by, a tube component as described with reference to FIGS. 1-4.

At operation 1510, the system provides a cable extending through the tube component of the apparatus. In some cases, the operations of this step refer to, or may be performed by, a cable as described with reference to FIGS. 4 and 13.

At operation 1515, the system provides a clamp adapter component of the apparatus attached to a first end of the tube component, where the clamp adapter component includes a clamp attachment part configured to attach the clamp adapter to a spinal clamp. In some cases, the operations of this step refer to, or may be performed by, a clamp adapter component as described with reference to FIGS. 3-5.

At operation 1520, the system provides an end assembly of the apparatus attached to a second end of the tube component, where the end assembly includes a tension adjustment part configured to adjust a tension of the cable. In some cases, the operations of this step refer to, or may be performed by, an end assembly as described with reference to FIGS. 3, 4, 8, and 9.

At operation 1525, the system provides a sheath component of the apparatus surrounding a portion of the tube component, where the sheath component includes a position locking mechanism configured to attach the sheath component to a base component. In some cases, the operations of this step refer to, or may be performed by, a sheath component as described with reference to FIGS. 3, 4, and 7.

At operation 1530, the system adjusts a tension of the cable using the tension adjustment part, where adjusting the tension of the cable modifies a rigidity of the tube component. In some cases, the operations of this step refer to, or may be performed by, a tension adjustment part as described with reference to FIGS. 4, 8, and 12.

At operation 1535, the system stabilizes a spine of a patient using the apparatus. In some cases, the operations of this step refer to, or may be performed by, a medical apparatus as described with reference to FIGS. 3 and 4.

At operation 1540, the system performs a medical procedure based on the stabilization. In some cases, the operations of this step refer to, or may be performed by, a medical processional as described in more detail herein.

Some examples of the method 1500 further include attaching the clamp adapter component to the spinal clamp. Some examples of the method 1500 further include attaching the sheath component to the base component, wherein the base component is fixed to a stationary surface.

The description and drawings described herein represent example configurations and do not represent all the implementations within the scope of the claims. For example, the operations and steps may be rearranged, combined or otherwise modified. Also, structures and devices may be represented in the form of block diagrams to represent the relationship between components and avoid obscuring the described concepts. Similar components or features may have the same name but may have different reference numbers corresponding to different figures.

Some modifications to the disclosure may be readily apparent to those skilled in the art, and the principles defined herein may be applied to other variations without departing from the scope of the disclosure. Thus, the disclosure is not limited to the examples and designs described herein, but is to be accorded the broadest scope consistent with the principles and novel features disclosed herein.

The described methods may be implemented or performed by devices that include a general-purpose processor, a digital signal processor (DSP), an application specific integrated circuit (ASIC), a field programmable gate array (FPGA) or other programmable logic device, discrete gate or transistor logic, discrete hardware components, or any combination thereof. A general-purpose processor may be a microprocessor, a conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration). Thus, the functions described herein may be implemented in hardware or software and may be executed by a processor, firmware, or any combination thereof. If implemented in software executed by a processor, the functions may be stored in the form of instructions or code on a computer-readable medium.

Computer-readable media includes both non-transitory computer storage media and communication media including any medium that facilitates transfer of code or data. A non-transitory storage medium may be any available medium that can be accessed by a computer. For example, non-transitory computer-readable media can comprise random access memory (RAM), read-only memory (ROM), electrically erasable programmable read-only memory (EEPROM), compact disk (CD) or other optical disk storage, magnetic disk storage, or any other non-transitory medium for carrying or storing data or code.

Also, connecting components may be properly termed computer-readable media. For example, if code or data is transmitted from a website, server, or other remote source using a coaxial cable, fiber optic cable, twisted pair, digital subscriber line (DSL), or wireless technology such as infrared, radio, or microwave signals, then the coaxial cable, fiber optic cable, twisted pair, DSL, or wireless technology are included in the definition of medium. Combinations of media are also included within the scope of computer-readable media.

In this disclosure and the following claims, the word “or” indicates an inclusive list such that, for example, the list of X, Y, or Z means X or Y or Z or XY or XZ or YZ or XYZ. Also the phrase “based on” is not used to represent a closed set of conditions. For example, a step that is described as “based on condition A” may be based on both condition A and condition B. In other words, the phrase “based on” shall be construed to mean “based at least in part on.” Also, the words “a” or “an” indicate “at least one.”

What is claimed is:

1. An apparatus comprising:
a tube component;
a cable extending through the tube component;
a clamp adapter component attached to a first end of the tube component, wherein the clamp adapter component comprises a clamp attachment part configured to attach the clamp adapter to a spinal clamp;
an end assembly attached to a second end of the tube component, wherein the end assembly comprises a tension adjustment part configured to adjust a tension of the cable; and
a sheath component surrounding a portion of the tube component, wherein the sheath component comprises a position locking mechanism configured to attach the sheath component to a base component.

2. The apparatus of claim 1, further comprising:
an additional tube component attached to the clamp adapter component;
an additional cable extending through the additional tube component; and
an additional end assembly attached to the additional tube component.

3. The apparatus of claim 2, wherein:
the cable extends through a first hole in the clamp adapter component, and the additional cable extends through a second hole in the clamp adapter component.

4. The apparatus of claim 1, wherein:
the clamp attachment part comprises a threaded screw hole.

5. The apparatus of claim 1, wherein:
the clamp attachment part comprises a plurality of teeth indentations configured to adjustably position the spinal clamp with respect to the clamp attachment part.

6. The apparatus of claim 1, wherein:
the tube component comprises a plurality of interlocking cylinders.

7. The apparatus of claim 6, wherein:
the each of the plurality of interlocking cylinders comprises a first end configured to attach to a second end of a subsequent interlocking cylinder.

8. The apparatus of claim 1, wherein:
the position locking mechanism comprises a curved extension configured to be inserted into a curved space within the base component.

9. The apparatus of claim 1, wherein:
the end assembly comprises a shell component comprising two interlocking halves.

10. The apparatus of claim 1, wherein:
the end assembly comprises a quick release nut and a spring-loaded locking rod.

11. The apparatus of claim 1, wherein:
the tension adjustment part comprises a threaded rod configured to adjust a tension of the cable when the threaded rod is rotated.

12. The apparatus of claim 1, further comprising:
a retaining disc on each end of the cable.

13. An apparatus comprising:
a plurality of tube components;
a plurality of cables, wherein each of the plurality of cables extends through a corresponding tube component of the plurality of tube components;
a clamp adapter component attached to a first end of each of the plurality of tube components, wherein the clamp adapter component comprises a clamp attachment part;
a plurality of end assemblies attached to a second end of each of the plurality of tube components, respectively, wherein each of the plurality of end assemblies comprises a tension adjustment part configured to adjust a tension of a corresponding cable of the plurality of cables; and
a plurality of sheath components surrounding a portion of each of the plurality of tube components, respectively, wherein each of the plurality of sheath components comprises a position locking mechanism.

14. The apparatus of claim 13, further comprising:
a spinal clamp attached to the clamp adapter, wherein the spinal clamp is configured to attach to a spine of a patient during a medical procedure.

15. The apparatus of claim 13, further comprising:
a base component attached to each of the plurality of sheath components using the position locking mechanism, wherein the base component is attached to a fixed surface.

16. A method comprising:
providing a tube component of an apparatus;
providing a cable extending through the tube component of the apparatus;
providing a clamp adapter component of the apparatus attached to a first end of the tube component, wherein the clamp adapter component comprises a clamp attachment part configured to attach the clamp adapter to a spinal clamp;
providing an end assembly of the apparatus attached to a second end of the tube component, wherein the end assembly comprises a tension adjustment part configured to adjust a tension of the cable; and
providing a sheath component of the apparatus surrounding a portion of the tube component, wherein the sheath component comprises a position locking mechanism configured to attach the sheath component to a base component.

17. The method of claim 16, further comprising:
attaching the clamp adapter component to the spinal clamp.

18. The method of claim 16, further comprising:
attaching the sheath component to the base component, wherein the base component is fixed to a stationary surface.

19. The method of claim 16, further comprising:
adjusting a tension of the cable using the tension adjustment part, wherein adjusting the tension of the cable modifies a rigidity of the tube component.

20. The method of claim 16, further comprising:
stabilizing a spine of a patient using the apparatus; and
performing a medical procedure based on the stabilization.
